SEQUENCE LISTING (6 sequences)

SEQ ID NO: 1 - DNA, 30 bp, Artificial Sequence
  5′ Primer for LTB construct
ccgctcgagaagctccccagtctattacag

SEQ ID NO: 2 - DNA, 33 bp, Artificial Sequence
  3′ primer for the LTB construct
cgcggatccctagttttccatactgattgccgc

SEQ ID NO: 3 - DNA, 36 bp, Artificial Sequence
  5′ primer for the VP2+5′Linker construct
gatccccgggtaccgagctcgacaaacctgcaagat

SEQ ID NO: 4 - DNA, 36 bp, Artificial Sequence
  3′ primer for the VP2+5′Linker construct
cccggaattctcgagttacggcacagctatcctcct

SEQ ID NO: 5 - DNA, 31 bp, Artificial Sequence
  5′primer for LTB+3′Linker construct
cgagaattcatggctccccagtctattacag

SEQ ID NO: 6 - DNA, 42 bp, Artificial Sequence
  3′ primer for the LTB1+3′Linker construct
cgagctcggtacccggggatcgttttccatactgattgccgc

(19) United States
(12) Patent Application Publication
(10) Pub. No.: US 2009/0304733 A1
(43) Pub. Date: Dec. 10, 2009

(54) Vaccine comprising recombinant ct or lt toxin

(76) Inventors: Jacob Pitcovski, Korazim (IL); Yelena Vasserman, Kiryat Shmona (IL); Elena Fingerut, Carmiel (IL)

Correspondence Address: BROWDY AND NEIMARK, P.L.L.C.;624 NINTH STREET, NW, SUITE 300, WASHINGTON, DC, 20001-5303, US

(21) Appl. No.: 11/658,847

(22) PCT Filed: Jul. 28, 2005
(86) PCT No.: PCT/IL05/00808

§ 371 Date: Jun. 22, 2009

(30) Foreign Application Priority Data
Jul. 28, 2004 (IL) 163249

(51) Int. Cl. A61K 39/116; C07K 14/245

(52) U.S. Cl. 424/192.1; 530/350; 424/203.1; 424/241.1; 424/236.1

(57) ABSTRACT

The present invention provides a recombinant toxin or the subunit B thereof selected from the group consisting of E. coli heat-labile enterotoxin (LT), its subunit B (LTB), cholera toxin (CT) and its subunit B (CTB), in immunogenic form, expressed in eukaryotic cells, a vaccine comprising said toxin or subunit B thereof, and use of said recombinant toxin or subunit B thereof in human or veterinary vaccines.

### FIELD OF THE INVENTION

The present invention relates to the production in eukaryotic cells of recombinant cholera toxin (CT) and E. coli enterotoxin (LT) and their B subunits CTB and LTB, respectively, and to their use as vaccines or as adjuvants in vaccines with various antigens.

ABBREVIATIONS: AOX1: alcohol oxidase I; AOX2: alcohol oxidase II; BMGY: buffered glycerol-complex medium; BMMY: buffered methanol-complex medium; CHO: Chinese hamster ovary; CMV: cytomegalovirus; CT: cholera toxin of Vibrio cholera; CTA: cholera toxin of Vibrio cholera subunit A; CTB: cholera toxin of Vibrio cholera subunit B; ETEC: enterotoxigenic E. coli; HF cells: high five cells; HRP: horseradish peroxidase; IBDV: infectious bursal disease virus; LT: heat-labile enterotoxin of Escherichia coli; LTA: heat-labile enterotoxin of Escherichia coli subunit A; LTB: heat-labile enterotoxin of Escherichia coli subunit B; MM: Minimal Methanol; rLTB: recombinant LTB; VP2: Viral protein 2; yrLTB: yeast rLTB.

### BACKGROUND OF THE INVENTION

Vaccination is the main method of protecting humans and animals against infectious diseases. In response to active vaccination, antibodies and memory B or T cells are produced which confer protection for long periods (on the order of years). Vaccines consist of the live, attenuated pathogen, the inactivated pathogen, or components of the pathogen. In the present era of genetic engineering, subunit vaccines are also being used, usually by producing a polypeptide of the pathogen in an expression system. Neutralizing antibodies to such a vaccine are induced upon injection of animals with an adjuvant (Liu, 1998).

The heat-labile enterotoxin of Escherichia coli (LT) and cholera toxin of Vibrio cholera (CT) cause two very serious diseases in developing countries. Both have similar pathogenic effects and show 95% sequence similarity (De Haan et al., 1999; Foss and Murtaugh, 1999), raising the possibility of using the LT or its subunit B molecule for vaccination against cholera. However, this molecule needs to be engineered in order to prevent the damage incurred by exposure to wild-type LT. Both toxins consist of five non-toxic B (CTB, LTB) subunits and one toxic A subunit, with a loop that is a central target for biological manipulation (Yamamoto and Yokota, 1983; Sixma et al., 1991; Yamamoto et al., 1984). The logical approach is therefore to use the non-toxic form instead of the native toxin.

Production of CTB or LTB without the A subunit has been attempted. CTB and LTB have been cloned and expressed in different expression systems, such as E. coli (L'Hoir et al., 1990; De Geus et al., 1997; Slos et al., 1994), Mycobacterium bovis (Hayward et al., 1999) and Lactobacillus or Bacillus brevis (Slos et al., 1998; Isaka et al., 1999; Goto et al., 2000), or surface-displaced on Staphylococcus xylosus and S. carnosus (Liljeqvist et al., 1997).

In recently published research, the CTB subunit was cloned into an E. coli host cell and anti-CT antibodies recognized the expressed protein. Moreover, the recombinant protein damaged the cells in vivo (De Mattos et al., 2002).

When LTB is expressed in genetically engineered bacterial cells, the product needs to be purified from its endotoxins. However, chemical purification of LTB from wild-type E. coli or of CT expressed in V. cholerae cultures may leave traces of the holotoxin (De Mattos et al., 2002).

Another important aspect is the immunostimulatory function of the LT/LTB, CT/CTB molecules, and the use of these molecules as adjuvants in vaccines (Ryan et al., 2001). This is based on LTB's potential to cause activation and differentiation of immune system cells (Williams et al., 2000). CTB and LTB have bean found to be effective adjuvants in co-administration (Isaka et al., 1999) and genetic or chemical fusion with antigens (Dertzbaugh et al., 1990).

LT and CT have been found to be effective mucosal adjuvants (De Haan et al., 1999; Walker et al., 1993; Rappuoli et al., 1999; Foss and Murtaugh, 1999; Liang et al., 1989; Ryan et al., 2001). LT and CT are both secreted toxins with similar sequence structure and activity, which cause diarrhea in humans (Spangler et al., 1992). LT is produced by enterotoxigenic E. coli (ETEC). Bacteria of this family produce two types of toxins, heat stable (ST) and heat labile (LT). The LT protein is composed of two subunits: the subunit A (LTA), a 28-kDa polypeptide, confers LT's toxicity. The 60-kDa subunit B (LTB) is composed of five identical polypeptides, which are synthesized separately with leader peptides for transfer to the cell periplasm. In the periplasm, the leader peptides are removed and a toxin unit is assembled by non-covalent linkage between one LTA and five LTBs (AB5) (Yamamoto and Yokota, 1983; Sixma et al., 1991; Spangler et al., 1992; Cheng et al., 2000; Yamamoto et al., 1984).

LTB, which has no toxic activity, is responsible for the binding of the toxin. It binds mainly to cellular receptors, GM1 gangliosides, but also, with lower affinity, to other gangliosides (Holmgren et al., 1985; Sugii and Tsuji, 1989; Spangler et al., 1992). Also CTB binds mainly to the receptor, GM1 ganglioside, on the surface of susceptible cells, and mediate the entrance of the toxin into the cells, whereby the A subunit, upon proteolytic activation, causes diarrhea.

CT and LT are immunogenic molecules that stimulate systemic and mucosal immune system responses (Hagiwar et al., 2001). However, the use of both CT and LT as an adjuvant is limited, partly because of the toxicity of CTA and LTA (Williams et al., 2000). Some studies have shown the importance of ADP-ribosyl transferase in the adjuvant activity of LT (Lycke et al., 1992; Feil et al., 1996). In the last decade, strategies have been developed to separate the adjuvant effect from the toxicity. Some researchers concluded that toxicity is part of the adjuvant effect, but others showed that the enzymatic effect of LTA is not essential for this purpose (Dickinson, 1995; De Haan et al., 1999; Douce et al., 1995; Douce et al., 1997; Giuliani et al.; 1998; Hagiwar et al., 2001; Lu et al., 2002).

The mechanism that enables adjuvant activity has not been elucidated. However, one report has found that the immunogenicity and adjuvant effects of LTB are dependent on its ability to bind to the cell receptor (most commonly GM1), whereas LT adjuvant characteristics are not dependent on binding to the receptor. This means that two independent mechanisms are involved in LT's enhancement of the immune response (de Haan et al., 1998; Ryan et al., 2001), and thus LTB can be used as an efficient carrier and adjuvant with no danger of toxification following vaccination. LTB has been found to activate specific signals in lymphocytes that induce selective activation and differentiation of those cells (Williams et al., 2000). The binding of LTB to GM1 was found to decrease the proliferation of mitogen-stimulated B cells on the one hand, and increase the expression of MHC class II and minor lymphocyte-stimulating determinants on the other (Francis et al., 1992). The effect of increasing MHC class II expression may explain the immunostimulatory effect of LTB.

Inactivated vaccines are injected intramuscularly or subcutaneously. Since most pathogens enter via mucosal tissues, an effective local response in these systems may block the pathogen. In order to activate such an immune response, antigen must be transferred to the mucosa and taken via dendritic cells to the peripheral lymph nodes, (McGhee et al., 1992; Boyaka et al., 1999; Ernst et al., 1999). Antibody level is the main parameter in such cases since this is the main way to neutralize toxins or pathogens (Ryan et al., 2001). Intranasal vaccination with CTB admixed with diphtheria toxoid elicits peripheral as well as systemic antibody responses (IgA and IgG, respectively) against the pathogen (Isaka et al., 1999). Similar results were found using LTB with influenza or bovine serum albumin (BSA) (Tochikubo et al., 1998). Moreover, following intranasal vaccination, antibodies were detected in other mucosal systems, such as the vagina (Verweij et al., 1998).

Addition of short polypeptides to the C-terminal or N-terminal end of LTB does not interfere with its tertiary structure or biological activity (Sandkvist et al., 1987; Schodel et al., 1989; Green et al., 1996; Sanchez et al., 1988; Dertzbaugh et al., 1990). Intranasal vaccination with peroxidase chemically linked to LTB induced a high level of anti-peroxidase antibodies in the sera, saliva, nasal fluids and lungs (O'Dowd et al., 1999).

Viral protein 2 (VP2) of infectious bursal disease virus (IBDV) of chicken has been found to induce the production of neutralizing antibodies when produced in a eukaryotic expression system (Pitcovski et al., 1996). This subunit vaccine was chosen as a model to show the potential of yeast-produced LTB for use as an adjuvant and carrier of subunit vaccines.

### SUMMARY OF THE INVENTION

It is the main object of the present invention to produce a recombinant protein selected from CT, LT, CTB, and LTB in eukaryotic cells, thereby eliminating the toxicity of bacterial endotoxins, while retaining the adjuvant effect of CT, LT, CTB and LTB for neutralizing antibodies induced by the recombinant toxin or subunit thereof.

The present invention thus provides a recombinant toxin or the subunit B thereof selected from the group consisting of E. coli heat-labile enterotoxin (LT), its subunit B (LTB), cholera toxin (CT) and its subunit B (CTB), in immunogenic form, wherein said immunogenic toxin or the subunit B thereof has been expressed in eukaryotic cells. In one preferred embodiment, the eukaryotic cells are yeast cells, more preferably, Pichia pastoris cells.

The recombinant toxins and subunits thereof can be used as vaccines against the respective bacteria or as adjuvants in vaccines with various antigens.

### BRIEF DESCRIPTION OF THE FIGURES

FIG. 1 shows LTB DNA fragment amplified by PCR. Lane 1: Molecular size markers; lane 2: LTB (310 bp).

FIG. 2 shows screening of Pichia pastoris colonies expressing recombinant LTB (rLTB) with specific anti-CT antibodies. 1-40—colonies transformed with LTB. 50, 51—colonies transformed with wild-type plasmid (negative control).

FIGS. 3A-3B show identification of rLTB expression in yeast by SDS-PAGE (A) or Western blotting (B). FIG. 3A: SDS-PAGE of induction medium stained with Coomassie blue to test expression of recombinant proteins. FIG. 3B: Immunoblot with anti-CT antibodies to detect rLTB protein expression during the induction. Lane 1: Commercial CTB protein; Lanes 2, 3, 4: Supernatant of yeast with wild-type plasmid at 5, 6, 7 days of induction, respectively; Lanes 5, 6, 7: Supernatant of yeast expressing rLTB at 5, 6, 7 days of induction, respectively. Lane 8: Molecular size marker. Samples were loaded on gel without boiling to avoid reduction of the pentamer structure into monomers.

FIG. 4 shows dot blot to test expression levels of rLTB in response to methanol concentration. Dots 1, 2, 3: rLTB expression following induction with 0.3%, 0.6% and 1.5% methanol, respectively; dots 4, 5: negative control—induction medium of wild-type transformed colony following induction with 0.3% or 1.5% methanol, respectively.

FIGS. 5A-5B are graphs showing rLTB protein purification by cation exchange chromatography. FIG. 5A: separation of induction medium of rLTB expressed in yeast. FIG. 5B: separation of induction medium wild-type plasmid expressed in yeast (negative control).

FIGS. 6A-6B: show purification of yeast rLTB (yrLTB) by cation-exchange chromatography tested by SDS-PAGE (A) and Western blotting (B). FIG. 6A: SDS-PAGE stained by Coomassie blue to test purification of yrLTB. FIG. 6B: Immunoblot with anti-CT antibodies to test purification of yrLTB. Lane 1: commercial CT protein; lanes 2, 3: elution fraction (38% NaCl) of yrLTB and wt plasmid respectively; lanes 4, 5: elution fraction (41% NaCl) of yrLTB and wt plasmid respectively; lanes 6, 7: fractions 2, 3 after boiling, respectively; lanes 8, 9: fractions 4, 5 after boiling, respectively. Broad arrow-pentamer of yrLTB protein. *-monomer of boiled yrLTB protein.

FIG. 7 shows DNA LTB-linker and VP2-linker fragments amplified by PCR—two first steps. Lane 1: molecular size markers; lane 2: LTB-linker (330 bp); lane 3: molecular size markers; lane 4: VP2-linker (1.42 kbp).

FIG. 8 shows LTB-VP2 DNA fragment amplified by PCR. Lane 1: molecular size markers; lane 2: LTB-VP2 (1725 bp).

FIGS. 9A-9B show identification of LTB-VP2 expression in yeast by SDS-PAGE (A) or immunoblot (B). FIG. 9A: lane 1: supernatant fraction of yeast with wild-type plasmid; lane 2: molecular size markers; lane 3: supernatant fraction of yeast expressing LTB-VP2. FIG. 9B: lane 1: supernatant fraction of yeast expressing LTB-VP2 detected by anti-CT antibodies; lane 2: supernatant fraction of yeast with wild type plasmid detected by anti-CT antibodies; lane 3: boiled supernatant fraction of yeast expressing LTB-VP2 detected by anti-CT antibodies; lane 4: boiled supernatant fraction of yeast with wild-type plasmid detected by anti-CT antibodies. *-monomer of LTB-VP2 (46 kDa); broad arrow—pentamer of LTB-VP2 (230 kDa).

FIG. 10 is an immunoblot to test for anti-CT antibodies in response to vaccination of broilers with rLTB expressed in yeast. The antigen CT (Sigma) was exposed to sera of birds vaccinated by: lane 1: commercial CT given orally; lane 2: induction medium of a colony expressing rLTB, given orally; lane 3: induction medium of a colony carrying wild-type plasmid, given orally; lane 4: commercial CT given by injection; lane 5: induction medium of colony expressing rLTB given by injection; lane 6: induction medium of colony carrying wild-type plasmid given by injection.

FIG. 11 is a graph showing anti-CT antibodies in broilers three weeks after vaccination with rLTB expressed in yeast, as determined by ELISA. Statistically significant differences (P<0.05) are indicated by an asterisk (*).

FIG. 12 is a graph showing antibody response in chicks, vaccinated with rLTB at 1 day of age. Statistically significant differences (P<0.05) are indicated by an asterisk (*).

FIG. 13 is a graph showing anti-IBDV antibodies three weeks after second vaccination with rLTB-VP2 expressed in yeast, as determined by ELISA. Statistically significant differences (P<0.05) are indicated by an asterisk (*).

### DETAILED DESCRIPTION OF THE INVENTION

The present invention provides, in one aspect, a recombinant toxin or the subunit B thereof selected from the group consisting of E. coli heat-labile enterotoxin (LT), its subunit B (LTB), cholera toxin (CT) and its subunit B (CTB), in immunogenic form, wherein said immunogenic toxin or the subunit B thereof has been expressed in eukaryotic cells.

For the sake of clarity, it is to be understood that the term “recombinant CT, LT, CTB or LTB”, as used herein, refers to recombinant CT, LT, CTB or LTB produced in eukaryotic cells.

Expression in eukaryotic systems, as opposed to prokaryotes, has three main advantages. The product is free of endotoxins, it is inexpensive and it allows production of fusion proteins that require post-translational modifications (e.g. glycosylation and phosphorylation) in order to be immunogenic and elicit the production of neutralizing antibodies. The resultant molecule may be used as a vaccine against the toxin itself or serve as an adjuvant in other vaccine.

According to one embodiment of the present invention, the eukaryotic system used is a yeast expression system. Yeast offer advantages over bacteria in heterologous protein production because, although they are unicellular organisms easy to manipulate and grow quickly, their cellular organization is eukaryotic, making it possible to perform expression and maturation processes characteristic of animal and plant cells. Moreover they can secrete recombinant proteins into the culture medium, being recombinant product levels higher there than in the cytoplasm. Even more, the secreted products are obtained with a high degree of purity (since few endogenous proteins are secreted) and therefore the purification steps are reduced. Finally, they offer a suitable environment for the adequate folding of proteins, especially of those that contain disulfide bonds.

In a preferred embodiment of the present invention, the methylotrophic yeast Pichia pastoris (P. pastoris) is used as the expression system. P. pastoris is a yeast that can metabolize methanol as the sole source of carbon and energy (methylotrophic) and is currently used for the production of recombinant proteins since, as a production system, it is simpler, cheaper and more productive than other higher eukaryotic systems. Being a yeast, it shares the advantages of easy genetic and biochemical manipulation of Saccharomyces cerevisiae but surpasses its heterologous protein production levels (10 to 100 times greater). According to the present invention, the CT, LT, CTB or LTB polypeptide can be produced by P. pastoris cells whose genome contains at least one copy of the cDNA sequence encoding said polypeptide, and under the regulation of a promoter region of a methylotrophic yeast gene that can be induced with methanol. The polypeptide product produced according to the present invention may be secreted into the culture medium in a high concentration.

In another embodiment of the present invention, CT, LT, CTB or LTB is expressed in mammalian cells. The recombinant DNA fragments encoding LT, CT, CTB or LTB are cloned into eukaryotic expression plasmids and transfected into mammalian cells for stable or transient expression. According to the invention, the preferred mammalian cells are Chinese hamster ovary (CHO) cells.

In a further embodiment of the present invention, LT, CT, CTB or LTB are expressed in insect cells through the baculovirus expression system. Recombinant baculoviruses are extensively used as vectors for abundant expression of foreign proteins in insect cell cultures. The appeal of the system lies essentially in easy cloning techniques and virus propagation combined with the eukaryotic post-translational modification machinery of the insect cell.

In preferred embodiments, the invention relates to the subunits B of LT and CT, and more preferably to LTB.

In another aspect, the invention provides a vaccine containing the recombinant LT, LTB, CT or CTB of the invention, more preferably LTB or CTB.

In one embodiment, the vaccine is a cholera vaccine containing the recombinant CT or CTB. In another embodiment, the vaccine is directed against E. coli heat-labile enterotoxin and contains the recombinant LT or LTB.

In a further aspect, the invention relates to the use of recombinant LT, LTB, CT or CTB, preferably produced in yeast cells, as an adjuvant in human or veterinary vaccines, and further provides a human or veterinary vaccine comprising the recombinant LT, LTB, CT or CTB of the invention and an antigen.

In one embodiment, the vaccine comprises a mixture of said recombinant LT, LTB, CT or CTB and said antigen. In another embodiment, the vaccine comprises said recombinant LT, LTB, CT or CTB chemically linked to said antigen. In a further embodiment, the vaccine comprises a fusion protein formed by said recombinant LT, LTB, CT or CTB and said antigen. In still a further embodiment, the said recombinant LT, LTB, CT or CTB can be co-administered with a human or veterinary vaccine.

The antigen for use in said vaccine of the invention may be any viral, bacterial, fungal or parasite antigen pathogenic to humans and/or to animals such as, but not limited to, antigens related to hepatitis A, B or C, or D virus, influenza virus, mouth and foot disease, cholera, rabies virus, herpes virus, human cytomegalovirus (CMV), dengue virus, respiratory syncytial virus, human papilloma virus, meningitis virus, Salmonella, Neisseria, Borrelia, Chlamydia, Bordetella, Streptococcus, Mycoplasma, Mycobacteria, Haemophilus, Plasmodium or Toxoplasma, Pasteurella multocida, etc.

The vaccines of the invention are intended both for human and veterinary use, and may be for oral, intranasal, mucosal, eye drop, vaginal, rectal transcutaneous or any other method of administration.

In one preferred embodiment, the invention provides a veterinary vaccine for poultry vaccination against infectious bursal disease virus (IBDV) containing recombinant LT, LTB, CT or CTB, preferably produced in yeast cells, and the IBDV VP-2 antigen, more preferably, as a fusion protein. In a most preferred embodiment, the IBDV vaccine comprises recombinant LTB produced in Pichia pastoris and the IBDV VP-2 antigen, preferably wherein the LTB and the VP-2 moieties are linked by a linker peptide. Viral protein 2 (VP-2) of IBDV of chicken was found to induce the production of neutralizing antibodies when produced in a eukaryotic expression system (Pitcovski et al, 1996). This subunit vaccine was chosen herein as a model to show the potential of yeast-produced LTB for use as an adjuvant and carrier of subunit vaccines.

In one embodiment, the present invention further relates to a recombinant fusion protein comprising LT, LTB, CT or CTB and an antigen that has to be expressed in eukaryotic cells, wherein said fusion protein has been expressed in eukaryotic cells, preferably yeast, cells. In one preferred embodiment, said recombinant fusion protein is expressed in the Pichia pastoris expression system.

In one embodiment, the recombinant fusion protein may comprise an antigen fused to LT via the B subunit of LT, or via the end of the A subunit (A1 domain) of LT. Also, the recombinant fusion protein may consist of a fusion protein in which the antigen substitutes the A1 domain of LT.

The antigen for use in said recombinant fusion protein may be any viral, bacterial, fungal or parasite antigen pathogenic to humans and/or to animals such as, but not limited to, antigens related to hepatitis A, B or C, or D virus, influenza virus, mouth and foot disease, cholera, rabies virus, herpes virus, human cytomegalovirus (CMV), dengue virus, respiratory syncytial virus, human papilloma virus, meningitis virus, Salmonella, Neisseria, Borrelia, Chlamydia, Bordetella, Streptococcus, Mycoplasma, Mycobacteria, Haemophilus, Plasmodium or Toxoplasma, Pasteurella multocida, etc.

In yet another aspect, the invention provides an isolated DNA molecule containing one or more copies of an expression cassette that includes:
(i) an alcohol oxidase promoter of a methylotrophic Pichia pastoris gene that can be induced with methanol;
(ii) a nucleotide sequence encoding LT, LTB, CT or CTB; and
(iii) an expression vector functional in Pichia pastoris.

The promoter region to be preferably used to lead the cDNA expression encoding the LT, LTB, CT or CTB polypeptide is derived from the P. pastoris alcohol oxidase gene inducible with methanol. This yeast is known to have two functional alcohol oxidase genes: alcohol oxidase I (AOX1) and alcohol oxidase II (AOX2). The coding regions of the two AOX genes are closely homologous, their restriction maps are alike and their amino acid sequence are very similar. The proteins expressed by the two genes have similar enzymatic properties, but the AOX1 gene promoter is more efficient with respect to its regulating function and renders higher levels of the gene product than the AOX2 gene promoter; its use is therefore preferred for LT, LTB, CT or CTB expression according to the present invention. The AOX1 gene, including its promoter, has been isolated and reported in U.S. Pat. No. 4,855,231.

The invention further provides a Pichia pastoris yeast cell comprising an expression vector that contains a nucleotide sequence encoding LT, LTB, CT or CTB, together with control elements enabling the expression of said nucleotide sequence in yeast host cells. In one preferred embodiment, the Pichia pastoris cell is transformed by homologous recombination with the DNA molecule above, particularly when the promoter and the termination sequence are from the Pichia pastoris AOX1 gene, wherein said DNA molecule integrates by homologous recombination into a Pichia pastoris which may use methanol as a sole carbon source. The transformed Pichia pastoris yeast cell may contain multiple copies of the expression cassette.

In a further embodiment, the invention relates to a viable culture of Pichia pastoris cells containing the transformed cells, and to a process for the production of a recombinant LT, LTB, CT or CTB polypeptide comprising culturing the Pichia pastoris cell culture under conditions wherein said polypeptide is expressed and, if desired, secreted into the culture medium. Preferably, the culture is grown in a medium containing methanol as a sole carbon source.

The invention further provides a recombinant LT, LTB, CT or CTB produced by the process as described herein above.

E. Coli enterotoxin (LT) and cholera toxin (CT) have been studied intensively as vaccines against these diseases and as adjuvants for mucosal vaccination. Two major problems interfere with the use of these promising molecules: their toxicity and the danger of other bacterial endotoxins being mixed in with the desired CT or LT. Expression of LTB or CTB by standard genetically engineered bacterial cells requires further purification of the product from the bacterial endotoxins. However, chemical purification of LTB from wild-type E. coli or of CT expressed in V. cholerae cultures may leave traces of the holotoxin (De Mattos, 2002).

The production of the recombinant toxins and, more particularly, of the recombinant B subunits CTB and LTB in eukaryotic cells according to the invention, eliminates the undesired endotoxins and enables the production of large quantities of LTB or CTB.

In one most preferred embodiment of the present invention, rLTB was expressed in P. pastoris host cells as a biologically functional protein. This expression system has three main advantages over bacterial expression systems. The first is that yeast cells do not produce endotoxins: because purification of endotoxins is an expensive process and it is hard to achieve totally pure samples, the use of yeast cells makes the purification process easier and the final product safer. Second, P. pastoris yeast cells are not pathogenic, even when administered live at very high concentrations (Pitcovski et al., 2003). Third, yeast is a eukaryotic organism that provides efficient and less expensive production of proteins as compared to expression in other eukaryotic systems. This system has been used for the production of various recombinant proteins.

The ability to produce recombinant protein genetically conjugated to LTB in a eukaryotic system enables the use of LTB as an adjuvant in cases in which the antigen should be expressed in such a system due to the need for glycosylation or other post-translational modifications. This was the case with the production of VP2, which provided protection only when expressed in a eukaryotic system. Another advantage of the yeast system is that the protein is secreted into the medium. The purification is simple and the fusion protein is in the correct form.

LTs have been found to be similar in sequence, immunological and physiochemical characteristics in various types of E. coli. In the present invention, the plasmid coding for LT was isolated from E. coli H10407, a strain that causes diarrhea in humans and is geographically widespread (Inoue et al., 1993). The LTB DNA fragment of the correct size (310 bp) (Sixma et al., 1991) was amplified by PCR and cloned into yeast cells. For comparison, the same gene was cloned in an E. coli expression system. High levels of pentameric protein were expressed in 20% of the yeast colonies. The protein was observed in the yeast culture supernatant, and identified by SDS-PAGE and immunoblotting with anti-CT antibodies. Moreover, yrLTB showed the natural biological activity of toxic LT—binding to the GM 1 receptor, and this activity disappeared following denaturation by boiling. Consequently, the LTB expressed in P. pastoris is probably in its correct native form. Since only the pentameric form of LTB can bind to the GM1 receptor (Liljeqvist et al., 1997), it may be assumed that yrLTB is correctly folded. This is crucial since the immunogenicity of LTB subunits is based exclusively on their ability to bind ganglioside receptors (De Haan et al., 1998; Green et al., 1996).

During the 3 last days of induction, the amount of yrLTB produced did not increase, but replacing the medium allowed its continuous expression in large quantities (data not shown). This might enable efficient scale-up for batch production of yrLTB. In E. coli, recombinant LTB or CTB were obtained in inclusion bodies in our studies (data not published) and others' (De Mattos et al., 2002), but the expression per cell was limited and the extraction complicated.

Most of the protein in the growth medium was the recombinant protein; however its concentration was relatively low. Cation-exchange chromatography enabled, in one step, purification and concentration of the yrLTB. Fusing a foreign polypeptide to yrLTB could result in changes in its folding. The current method was performed under native conditions and was based on the isoelectric point of the recombinant protein, avoiding changes in protein folding during separation. The purified yrLTB was obtained at high concentrations and showed biological activity similar to that observed prior to being run through the column.

Antibodies play a major role in neutralizing bacterial toxins and preventing adherence to surface receptors on host cells r-LTB was found to be an immunogenic molecule. Injection of 3 μg rLTB elicited the secretion of specific antibodies. Following oral administration of 3 μg of rLTB, the recombinant protein successfully traversed the digestive system and an effective systemic antibody response was detected. For comparison, the effective oral dose of BSA to activate an antibody response against it is 25 mg per bird per day for six days (Klipper et al., 2000). In previous studies, a low amount of CT elicited an immune response via oral administration. This was related to its hyper-immunostimulatory effect, its ability to withstand digestion by proteolytic enzymes and its ability to penetrate intestinal cells. Recombinant LTB appears to have similar qualities. This ability of yrLTB to cross the digestive tract raises the possibility of fusing a protein to it and performing oral vaccinations. Another interesting finding is that by the oral route, an immune response could be achieved at as early as 1 day of age, in contrast to intramuscular injection which did not induce any antibody response at that age.

The ability to enhance the immune response against a protein that is fused to yrLTB was tested with VP2. This protein is used as a commercial subunit vaccine in poultry and confers protection against IBD when produced correctly in eukaryotic organisms upon administration with oil adjuvant (Pitcovski et al., 2003). No antibodies against LTB were found, leading us to conclude that the LTB pentamer is surrounded by five VP2 proteins, so anti-CT antibodies cannot bind. However, vaccination with rLTB-VP2 conferred full protection against challenge with virulent IBDV. In previous studies, injection of a protein without an adjuvant did not lead to the production of neutralizing antibodies at protection levels.

According to the present invention, we have seen that immunization by VP2 via eye-drops, without adjuvant, did not initiate antibody secretion, whereas yrLTB as a fusion protein with VP2 yielded an immune response to VP2 after only one vaccination. Thus, the results of this experiment proved the adjuvant effect of yrLTB. yrLTB enhanced antibody production against some of the antigens that were co-administered by intramuscular injection. No antibodies were detected to a co-administered protein given orally (data not shown). Therefore, the advantage of a fused molecule is that it may allow oral vaccination.

In a safety study in mice, it was found that intranasal administration of CT may target neuronal tissue and may promote uptake of vaccine proteins into olfactory neurons in addition to nasal-associated lymphoreticular tissues (van Ginkel et al., 2000). In other studies, mutant LT was found to be an effective and safe adjuvant for nasal immunization vaccine (Hagiwar et al., 2001). Clinical safety of LT delivered transcutaneously was tested in adult volunteers and the vaccine was found to be safe and effective (Guerena-Burgueno et al., 2002).

In summary, high levels of purified yrLTB were expressed in P. pastoris yeast cells and were secreted into the culture medium. The protein was purified and concentrated and was found to bind to GM1 ganglioside. When administered by injection or orally to birds, high anti-LTB antibody titers were produced. It was further found to be efficient as an adjuvant. The adjuvant quality of yrLTB was proven by co-administration with, or fusion to, antigens. Thus, this efficiently produced and purified molecule can safely be used for vaccination against the toxin itself or as a carrier for a foreign vaccine molecule.

The invention will now be illustrated by the following non-limiting examples.

### EXAMPLES

### Materials and Methods

(i) Cloning the LTB Gene in the Yeast Pichia pastoris

E. coli plasmid H10407 (Yamamoto and Yokota, 1983; Inoue et al., 1993) was used as a template to synthesize the LTB fragment. The DNA sequence of the fragment encoding the B subunit of LT was propagated by PCR using primers of the ends of the gene as published previously (GenBank accession number AB011677). Sequence of primers: 5′ primer for the LTB construct: 5′ ccg ctc gag aag ctc ccc agt cta tta cag 3′ (SEQ ID NO: 1), 3′ primer for the LTB construct: 5′ cgc gga tcc cta gtt ttc cat act gat tgc cgc 3′ (SEQ ID NO: 2).

The reaction solution contained 1 unit of Taq polymerase (Promega, Madison, Wis., USA), 5 μl of Taq buffer (20 mM Tris-HCl pH 8.3, 50 mM KCl, 2 mM MgCl₂) and 20 pmol of each primer in a final volume of 50 μl. The PCR scheme was as follows: 5 min at 94° C., 2 min at 42° C., 60 s at 74° C., (60 s at 94° C., 2 min at 50° C., 3 min at 74° C.)×28 cycles, 15 min at 74° C. The PCR product was electrophoresed in a 1% agarose gel and visualized by ethidium bromide staining.

The DNA fragment was purified with a “PCR products purification system” kit (GibcoBRL, UK) and cloned into a pHILSI plasmid (Invitrogen, San Diego, Calif., USA) using the XhoI and BamHI restriction sites incorporated into the primers. The recombinant plasmid was transformed into Top10 E. coli cells (Invitrogen) according to the manufacturer's instructions.

The colonies that grew on LB plates containing ampicillin (100 μg/ml) were screened for the LTB fragment by PCR. The plasmid from the positive colonies was purified by mini-preps kit (Promega) according to manufacturer's instructions, and tested again with restriction enzymes. The DNA sequence of colonies carrying the desired gene was determined (Hebrew University, Biotechnology Services, Jerusalem, Israel).

To promote integration into the AOXI locus of the yeast genome, the recombinant plasmid was linearized with BglII restriction enzyme. The linearized r-plasmid was cloned into P. pastoris SMD1168 (Invitrogen) according to kit instructions as recommended by the manufacturer in: ‘A Manual of Methods for Expression of Recombinant Proteins in Pichia pastoris’, Version 1.8.

(ii) Screening for Pichia pastoris Colonies Expressing LTB

All growth media and plates were prepared according to the manufacturer's recommendations (Invitrogen).

The recombinant colonies were grown on Regeneration Dextrose Base plates for 4 days at 30° C. A hybond-C nitrocellulose membrane (Amersham International, Little Chalfont, UK) was placed over the Minimal Dextrose (MD) plate and single colonies were pasted onto the membrane. A colony transfected with a wild-type plasmid was grown as a negative control.

After 2 days at 30° C., the membrane was transferred onto a Minimal Methanol (MM) plate. Following 7 days' incubation at 30° C., 100 μl methanol was added twice a day to induce protein production. The same colonies were grown and kept as master stocks.

The membrane was washed three times, 10 min each, in TNT buffer (150 mM NaCl, 10 mM Na₂HPO₄, 10 ml Tris-HCl pH 8.0, 0.05%, v/v, Tween 20) and blocked with milk buffer (150 mM NaCl, 10 mM Na₂HPO₄, 10 mM Tris-HCl pH 7.0 in milk, 1%, w/v, fat) for 30 min. After two additional 10-min washes in TNT buffer, the membrane was incubated with a 1:1000 dilution of rabbit anti-CT polyclonal antibody (rabbit anti-CT, Sigma, St. Louis, Mo., USA) at 37° C. for 1 h. The membrane was washed twice with TNT buffer and incubated with a secondary antibody—horseradish peroxidase (HRP)-conjugated goat anti-rabbit IgG (Sigma), under the same conditions. Following two washes, the membrane was exposed to the HRP substrate solution 3,3′-diaminobenzidine (Sigma) until color developed. The stained colonies were isolated and stored at 4° C. for further growth and expression.

(iii) Expression of Recombinant LTB in P. pastoris

All colonies that were detected by anti-CT were grown in 50 ml buffered glycerol-complex medium (BMGY) at 30° C. for 48 h until the OD₆₀₀ₙₘ reached 10 to 20. The cells were centrifuged for 10 min at 6000 RPM and resuspended in 10 ml buffered methanol-complex medium (BMMY). At this point the expression of recombinant (r) LTB was induced by the addition of 300 μl methanol (to a final concentration of 0.3%, v/v) every 12 h. The optimal period of induction was determined by collecting samples on each day of induction. The samples were centrifuged as previously. The supernatant, containing the soluble yeast (y) rLTB, was collected and stored at 4° C.

(iv) Protein and Antibody Assays—SDS-PAGE and Western Blotting rLTB protein—The supernatant containing the soluble yeast rLTB was analyzed by SDS-polyacrylamide gel electrophoresis (PAGE) and immunoblot (Western blot). Loading buffer (3%, w/v SDS and 5%, v/v mercaptoethanol) was added to each sample, which was then boiled, or not, and electrophoresed in 12% polyacrylamide slab gels, using a discontinuous SDS gel system (Bio Rad, Hercules, Calif., USA). In most cases, two slab gels were electrophoresed simultaneously. One was stained with Coomassie Brilliant Blue R, and the proteins from the second were electrotransferred onto a nitrocellulose filter (Hybond C, Amersham International) using a semi-dry system (Bio Rad), and Western blotting was performed. Following blocking with milk buffer, the membrane was incubated for 1 h at 37° C. with rabbit anti-CT antibody (Sigma) diluted 1:1000 in milk buffer. Filters were washed twice in PBS and incubated with goat anti-rabbit IgG-peroxidase conjugate (Sigma) diluted 1:1000, followed by incubation with the substrate solution 3,3′-diaminobenzidine (Sigma). One positive clone was chosen to continue the experiment.

Testing antibodies—CT was added to sample buffer (3% (w/v) SDS and 5%, (v/v), mercaptoethanol) and electrophoresed in 12% polyacrylamide slab gels, using a discontinuous SDS gel system (Bio Rad). The CT was electrotransferred onto a Hybond C nitrocellulose filter using a semi-dry system (Bio Rad), and the filter was incubated for at least, 1 h, in milk buffer.

The filter was cut into 5-mm wide strips and then incubated separately for 1 h in the relevant sera diluted 1:200 in dilution buffer (PBS with 0.05% BSA). After several washes in PBS, the filters were incubated with rabbit anti-chicken IgG-peroxidase conjugate (Sigma) diluted 1:1000 in dilution buffer, followed by incubation in the substrate solution 3,3′-diaminobenzidine.

### (v) Protein and Antibody Assays—Functional Assay—ELISA

Recombinant protein and antibodies produced in response to vaccination with yrLTB were determinated by ELISA (enzyme-linked immunosorbent assay).

Testing the ability of the recombinant protein to bind to GM 1 receptor. Each of the following steps was followed by three washes in PBS buffer with 0.05% Tween 20 and drying on a paper towel.

ELISA plates were incubated for 2 h at 37° C. or overnight at 4° C. with GM1 receptor (monosialoganglioside-GM1, Sigma) diluted in carbonate-coating buffer (pH 9.6) to a final concentration of 15 μg/ml. Skim milk in PBS (1:1, v/v) was added for 1 h at 37° C. as a blocking step. The supernatant being tested was incubated for 1 h at 37° C. and rabbit anti-CT antibody diluted 1:1000 in PBS buffer was used to detect the protein. This was followed by incubation with a secondary antibody, goat anti-rabbit IgG conjugated to HRP diluted 1:1000 in PBS buffer. A substrate solution, o-phenylenediamine dihydrochloride (Sigma), was added and OD was determined by ELISA READER (Lumitron) at 450 nm.

Test recognition of CT by anti-rLTB. Each of the following steps was followed by three washes in PBS buffer with 0.05% Tween 20 and drying on a paper towel.

ELISA plates were incubated for 2 h at 37° C. or overnight at 4° C. with GM1 receptor diluted in carbonate-coating buffer (pH 9.6) to a final concentration of 15 μg/ml. Skim milk (50%) in PBS was added for 1 h at 37° C. as a blocking step. Commercial CT (Sigma) diluted 1:1000 in PBS buffer and used as an antigen, was bound to the GM1 receptor for at least 1 h, at 37° C. The sera being tested was diluted 1:500 in PBS buffer and incubated for 2 h at 37° C. This was followed by incubation with a secondary antibody, rabbit anti-chicken IgG-conjugated HRP diluted 1:1000 in PBS buffer o-Phenylenediamine dihydrochloride was added and OD was determined by ELISA READER at 450 nm.

### (vi) Optimizing Protein Expression.

The selected clone was grown in 150 ml of BMGY to an OD₆₀₀ of 10 to 20. The culture was centrifuged for 10 min at 6000 RPM. The harvested cells were resuspended in 30 ml of BMMY, divided into three tubes, 10 ml per tube, and grown for 7 days. LTB expression was determined by adding of methanol at concentrations of 0.3, 0.6 and 1.5% twice daily. On days 5, 6 and 7, samples of induction medium were collected. A dot blot was used to detect LTB protein using rabbit anti-CT antibody and goat anti-rabbit-HRP antibody.

(vii) Purification and Concentration of Yeast rLTB Protein.

Ion-exchange chromatography was used for purification of the yrLTB protein. A strong cation-exchange resin, Macro-Prep High S Support (Bio Rad), in AKTA prime device (Amersham Pharmacia Biotech), was used for rLTB purification. To adsorb yrLTB to the resin, the induction medium with the protein was diluted 1:10 in DDW. Following adsorption of the protein, the column was washed with 10 volumes of binding buffer (25 mM sodium phosphate, pH 6.8). rLTB was eluted in a linear NaCl gradient with increasing additions of elution buffer (25 mM sodium phosphate, 1 M NaCl, pH 6.8). yrLTB concentration in the eluates was calculated from the BSA curve produced by Bradford test.

(viii) Cloning of the LTB-VP2 Fusion Gene in P. pastoris and Expression of the Recombinant Protein

The 5′ terminus of the VP2 gene was genetically fused to the 3′ terminus of the LTB gene. The fusion gene LTB-VP2 was constructed by three-step PCR. A seven-amino-acid, proline-containing linker (Clements. 1990) was included between the LTB and VP2 moieties. The DNA sequences of fragments encoding LTB and VP2 were isolated by the two first PCR steps using primers of the 5′ and 3′ ends of each gene (GenBank accession numbers AB011677 and L42284, respectively). The reactions were performed as described earlier (Materials and Methods, section i).

5′ primer for the VP2+5′Linker construct:

(SEQ ID NO: 3)

5′-gat ccc cgg gta ccg agc tcg aca aac ctg caa
gat-3′,

3′ primer for the VP2+5′Linker construct:

(SEQ ID NO: 4)

5′-ccc gga att ctc gag tta cgg cac agc tat cct
cct-3′,

5′primer for LTB+3′Linker construct 5′-cga gaa ttc atg gct ccc cag tct att aca g-3′ (SEQ ID NO: 5), 3′ primer for the LTB1+3′Linker construct 5′-cga gct cgg tac ccg ggg atc gtt ttc cat act gat tgc cgc-3′ (SEQ ID NO: 6).

The DNA fragments were purified with GibcoBRL's “PCR products purification system” and used as a template for synthesis of the full fusion protein in the third PCR step (with primers of SEQ ID NO: 3 and SEQ ID NO: 6). The PCR scheme was as described earlier (Materials and Methods, section i). The PCR products were electrophoresed in an agarose gel and visualized by ethidium bromide staining.

Following purification with the same kit, the fusion DNA fragment was cloned into the P. pastoris plasmid pHILD2 (Invitrogen) using restriction sites incorporated into the primers. The recombinant plasmid was transformed into Top10 E. coli cells according to the manufacturer's instructions. Screening of bacterial colonies was performed as described previously, and the NotI-linearized r-plasmid was cloned into P. pastoris GS1168 according to kit instructions.

Yeast colonies expressing LTB-VP2 protein were screened as described in Pitcovski et al. [submitted to Vaccine Journal, 42]. Briefly, the yeast colonies were placed on a Hybond-C nitrocellulose membrane and grown on MD plates for 2 days at 30° C., and then transferred with the membrane onto MM plates. Following 5 days of induction, the yeast colonies were lysed by yeast lytic enzyme (ICN, Costa Mesa, Calif., USA) and expressed protein was recognized by anti-CT or anti-IBDV (ABIC, Jerusalem, Israel) antibodies.

The MGY medium was inoculated with the selected colony and incubated at 30° C. to an OD₆₀₀ of 1 to 2. The culture was centrifuged and resuspended in MM medium. rLTB-VP2 production was induced by the addition of methanol every 12 h for 5 days. Following induction, the cells were broken by vortexing with glass beads, centrifuged, and the supernatant contained the soluble rLTB-VP2. Supernatant was analyzed by SDS-PAGE and immunoblot using anti-CT antibody or polyclonal anti-IBDV antibody.

### (ix) Immunogenicity of Recombinant LTB Protein in Chickens

Two sets of experiments were carried out, one on laying hens and broilers, and the second on one-day-old chicks. Six groups of six birds each were included in each experimental trial. Birds were treated twice at a 3-week interval. Birds were vaccinated intramuscularly or orally with 3 μg of rLTB. CT (50 μg) was used as a positive control and the supernatant from wild-type plasmid was used as a negative control. No adjuvant was added to the experimental vaccines. Blood was drawn 3 weeks after the second vaccination and sera were kept at −20° C. until use. The presence of antibodies in the sera was tested by Western blotting and ELISA, as described previously.

The second set of experiments was comprised of four groups with five chicks in each. Three groups were vaccinated orally, intramuscularly or by eye-drops with 17 μg of purified yrLTB, without adjuvant. The fourth untreated group served as a negative control. Blood was drawn two weeks post vaccination and sera were stored at −20° C. until use. Antibody levels against LT were tested by ELISA as previously described.

### (x) Immunogenicity of Recombinant LTB-VP2 Protein in Chickens

Two experiments were conducted to test rLTB-VP2. In one experiment, five groups of 10 birds each were tested. Birds, at the age of 5 weeks, were vaccinated twice at a 3-week interval intramuscularly with 150 or 30 μg of rLTB-VP2, or orally with 150 μg of rLTB-VP2. The commercial vaccine (Bursative 2, ABIC) against IBDV, containing VP2 in adjuvant, was used as a positive control and lysate of wild-type plasmid was used as a negative control. Blood was drawn 3 weeks after each vaccination. The presence of antibody was tested by agar gel precipitation (AGP) and ELISA using CT and IBDV as antigens as described previously. IBDV challenge was performed 3 weeks after the second vaccination as previously described (Pitcovski et al., 1996).

In the second experiment, three groups of three chicks each were vaccinated via eye-drops with 50 μg yrLTB in 1001 sodium phosphate buffer, or 50 μg of LTB-VP2 fusion protein in 2001 sodium phosphate buffer, or VP2 commercial vaccine as a positive control. No adjuvant was added to the experimental vaccines. Blood was drawn 2 weeks after vaccination and sera were stored at −20° C. until use. Antibody levels in the sera were tested by ELISA using CT and VP2 as antigens, as described previously.

### Example 1

### Extraction of the Plasmid from E. coli and Cloning the LTB Gene in the Yeast

Three plasmids were extracted from E. coli. H10407 (Inoue et al., 1993). Two bands were identified in the agarose gel, the smaller one carrying the LTB gene.

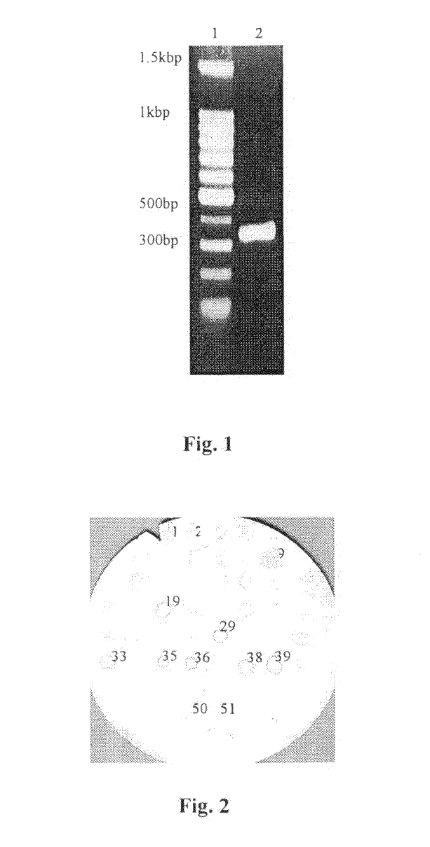

The open reading frame of LTB from a plasmid extracted from E. coli was amplified by PCR using oligonucleotides corresponding to both ends of the desired gene, as described in Materials and Methods. The PCR product was electrophoresed in a 1% agarose gel and visualized by ethidium-bromide staining (FIG. 1). One sharp band of amplified LTB DNA could be seen at the expected size (approximately 310 bp). The DNA fragment was extracted and cloned into E. coli, and PCR, restriction analysis and DNA sequencing confirmed correct cloning of the LTB. Following amplification of the recombinant plasmid, the construct was cloned into yeast cells.

### Example 2

### Screening for Pichia pastoris Colonies Expressing LTB

Following 7 days of methanol induction, the nitrocellulose filter carrying the yeast-colony proteins was probed with specific antibodies (FIG. 2). The screening method for expressing yeast colonies, which was developed in the laboratory of the present inventors, allows direct identification of colonies expressing the desired protein. A clearly visible circle appeared in some of the colonies (19, 29, 33, 35, 36, 38 and 39) but not in the negative control (colonies 50 and 51). About 15% of the colonies were found to express yrLTB.

### Example 3

### Expression and Purification of yrLTB in Yeast Culture

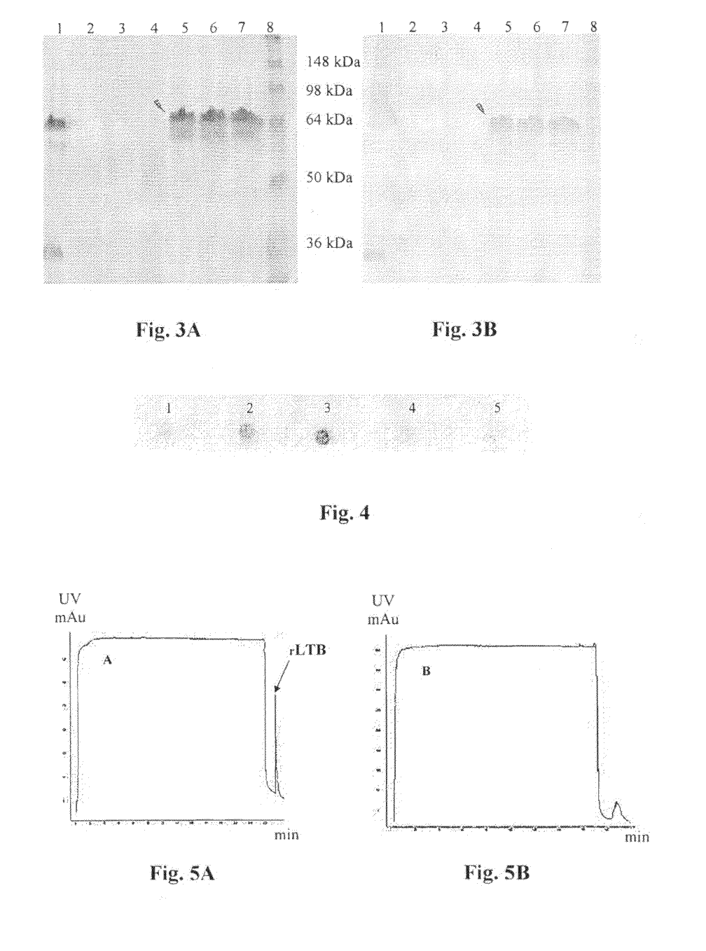

rLTB production was induced in all positive colonies (1 ml culture) and screened for yield. The colony yielding the highest protein expression was chosen for further experiments. Supernatant samples of the selected colony were collected on days 5, 6 and 7 of incubation and analyzed by SDS-PAGE (FIG. 3A) and Western blotting (FIG. 3B). Pentameric yrLTB was seen on all days and identified by specific antibodies against CT (lanes 5-7). No bands were found in the negative control.

The expressed protein was tested by ELISA and found to bind to the ganglioside receptor GM1. The results shown in Table 1 indicate that LTB was in the correct pentameric form. Boiling yrLTB for 5 to 10 min caused denaturation of the pentameric structure and almost completely abolished GM1 binding.

TABLE 1
Results of an ELISA testing for rLTB's ability to bind the
ganglioside receptor GM1.

| | OD_{450 nm} | | | | | |
|---|---|---|---|---|---|---|
| | Yeast | WT yeast | Bacterial | WT yeast | | |
| Antigen | rLTB | plasmid | rLTB | plasmid | ELISA controls | |
| 37° C. | 1.53 | 0.28 | 1.58 | 0.30 | CT as an | 1.66 |
| | | | | | antigen | |
| Boiled | 0.16 | 0.14 | 0.33 | 0.26 | without Ag | 0.26 |
| | | | | | without AbI | 0.05 |

WT—wild type;
CT—cholera toxin;
Ag - antigen;
AbI - rabbit anti-CT antibody

While attempting to improve yrLTB expression, we found that the level of expression is proportional to the methanol concentration during the induction (FIG. 4). Each sample (70 μl) was loaded onto a nitrocellulose membrane and exposed to specific antibodies. The maximal yield of yrLTB was obtained at 7 days' induction with 1.5% methanol. These optimal conditions were used for large-volume induction of yrLTB protein for subsequent in vivo experiments.

### Example 4

### Purification and Concentration of Yeast rLTB Protein

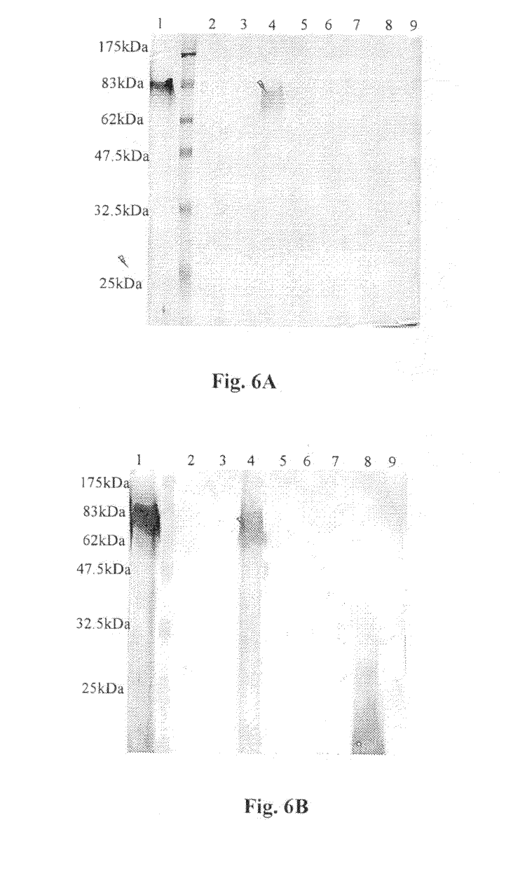

The pentameric LTB protein is a strong cation. yrLTB was purified by cation-exchange chromatography (FIG. 5). Binding to the resin was performed under neutral pH conditions and elution was affected by a NaCl continuous gradient. SDS-PAGE (FIG. 6A) and Western blotting (FIG. 6B) confirmed the purification. A strong band of pentameric yrLTB, or monomeric yrLTB after boiling, could be seen in the elution fraction (lane 4 and lane 8, respectively).

To confirm these results, fractions from different point in the separation process were tested by ELISA for their ability to bind to GM1 receptor. The results are shown in Table 2. No rLTB was identified in the wash fraction, but high titers were identified in three of the eluted fractions.

TABLE 2
ELISA to test the ability of purified rLTB proteins to bind
ganglioside receptor GM1.

| Fractions | | Fractions | | | | |
|---|---|---|---|---|---|---|
| before | | transferred | | | | |
| separation | | column | | Elution fractions | | |
| OD_{450 nm} | | OD_{450 nm} | | OD_{450 nm} | | |
| | wt | | wt | | | wt |
| RLTB | plasmid | rLTB | plasmid | rLTB | % NaCl | plasmid |
| 1.19 | 0.11 | 0.29 | 0.12 | 0.09 | 38% | 0.10 |

| | | | | | |
|---|---|---|---|---|---|
| Commercial | 0.26 | 0.12 | 1.11 | 42% | 0.15 |
| CT-1.50 | | | | | |
| without | 0.24 | 0.09 | 1.21 | 49% | 0.14 |
| AbI-0.08 | | | | | |
| | 0.23 | 0.09 | 1.22 | 54% | 0.08 |
| | | | 0.78 | 60% | 0.10 |
| | | | 0.38 | 66% | 0.08 |

### Example 5

### Expressing yrLTB-VP2 Fusion Protein in P. pastoris

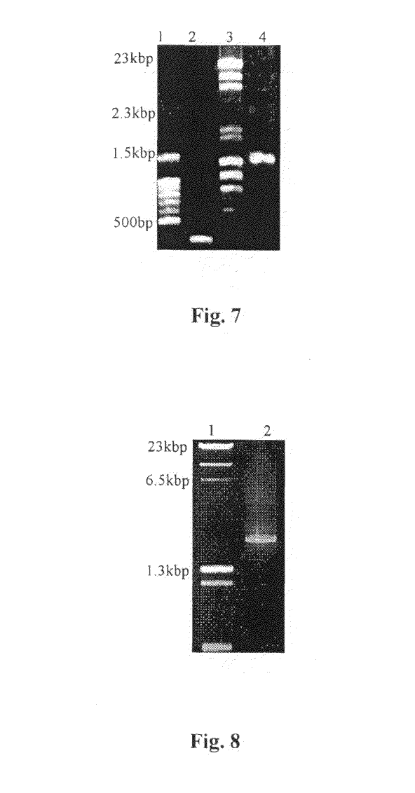

The DNA fragments of LTB and VP2 with the proline linker at the 3′ and 5′ terminus, respectively, were amplified by PCR. The PCR products (FIG. 7, FIG. 8) were used as templates to synthesize the fusion gene. The DNA fragment was cloned into P. Pastoris plasmid. The recombinant plasmid was transformed into E. coli, followed by cloning into P. pastoris host cells.

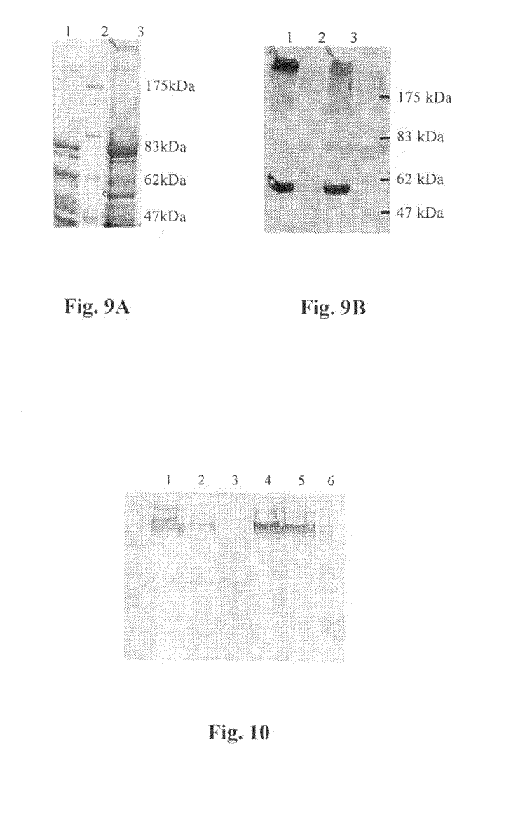

Expression of the recombinant protein in P. pastoris was induced by methanol and the cells were disrupted by glass beads and centrifuged. yrLTB-VP2 was found in the supernatant and analyzed by SDS-PAGE (FIG. 9A) and Western blot (FIG. 9B). Both pentameric and monomeric forms of the yrLTB-VP2 fusion protein appeared at the expected sizes. The boiled, denatured recombinant protein was recognized by anti-CT (FIG. 7B) and anti-IBDV antibodies (data not shown).

The expressed protein was tested by ELISA and found to bind GM1 (Table 3). It should be pointed out that yrLTB was in the correct pentameric structure, and fusion of a foreign protein to its 3′ terminus did not change its folding. The native form of yrLTB-VP2 was recognized by anti-IBDV, but not by anti-CT. Recognition by the former indicates correct folding of the VP2 protein.

TABLE 3
Results of ELISA testing the ability of LTB-VP2 to bind the
ganglioside receptor GM1.

| | Anti-CT antibodies | | | Anti-IBDV antibodies | | |
|---|---|---|---|---|---|---|
| | LTB- | WT | Commercial | LTB- | WT | Commercial |
| OD_{450 nm} | VP2 | plasmid | CT | VP2 | plasmid | CT |
| 37° C. | 0.38 | 0.52 | 1.82 | 1.21 | 0.60 | 0.60 |
| Boiled | 0.40 | 0.44 | 0.6 | 0.67 | 0.66 | 0.68 |

| | | |
|---|---|---|
| Without | 0.46 | 0.60 |
| Ag | | |
| Without | 0.35 | 0.60 |
| AbI | | |

WT—wild type;
CT—cholera toxin;
Ag - antigen;
AbI - rabbit anti-CT antibody.

### Example 6

### Immunogenicity of Recombinant LTB Protein in Chickens

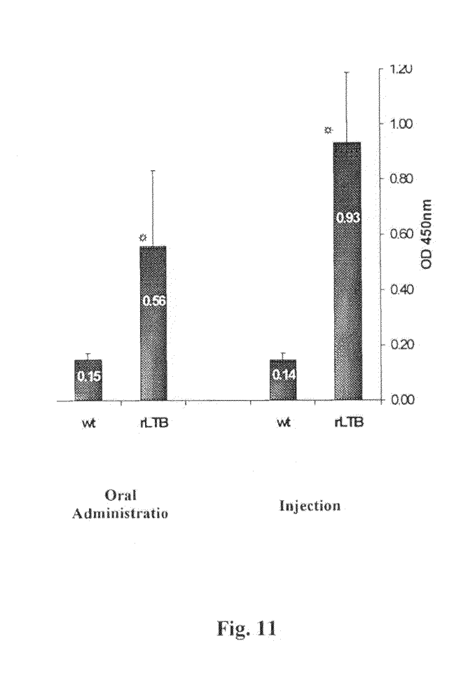

The expressed yrLTB protein was injected intramuscularly or administered orally to broilers. Blood was taken 3 weeks after the second vaccination. The ability of yrLTB to elicit an immune response and to induce antibody secretion was tested by Western blotting (FIG. 10) and ELISA (FIG. 11). According to the ELISA results, all six injected birds and five of their orally vaccinated counterparts produced antibodies that recognized commercial CT.

The laying hens showed a similar response to the vaccination. The difference in antibody level between the experimental group and the negative control was significant.

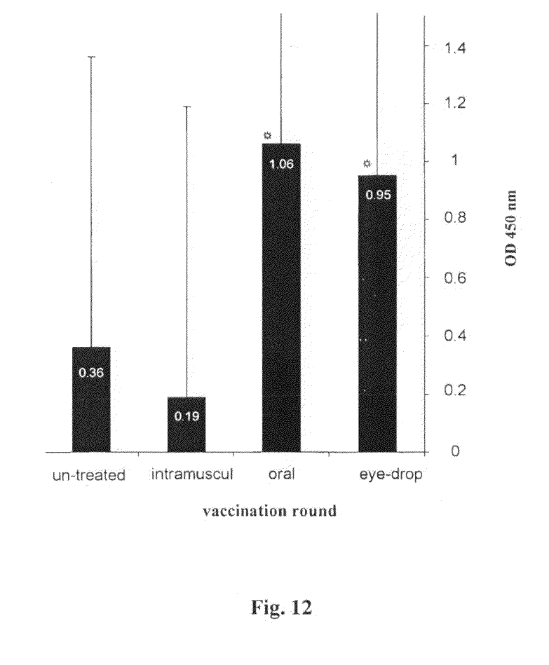

One-day-old birds vaccinated with yrLTB, orally or via eye-drops, showed an antibody response. No antibodies were detected by ELISA in birds vaccinated intramuscularly (FIG. 12).

### Example 7

### The Adjuvant Effect of yrLTB Protein in Turkeys

The ability of yrLTB to increase the response against the Pasteurella multocida type 3 (Pm3) vaccine was tested. The experiment included three groups, 14 turkeys per group, which were vaccinated twice at a 3-week interval, followed by challenge with pathogenic P. multocida bacteria (95 cfu per poult). The tested groups were intramuscularly injected with 0.05 ml of inactivated Pm3 in emulsion and 2 to 3 μg yrLTB. Pm3 bacteria in commercial water-in-oil adjuvant was used as a positive control and the PBS buffer was used as a negative control. The rLTB was intramuscularly injected as an adjuvant for fowl cholera (“cholerin”) vaccine. The ability of the recombinant protein to increase the response to the vaccine was tested by challenging the birds with pathogenic bacteria. The results are shown in Table 4. The addition of rLTB increased the response by 14% relative to negative-treated birds, to the same protection level as the positive control.

TABLE 4
The adjuvant effect of rLTB in protection against
virulent cholerin following two vaccinations.

| Type of vaccination | % Hedelston | % of post-challenge death |
|---|---|---|
| Pm3 + rLTB + w/o | 93 | 1/14 |
| PBS + rLTB + w/o | 79 | 3/14 |
| Positive control | 93 | 1/13 |

% Hedelston is an indicator for protection against cholerin. Up to 60% is regarded as positive response.

### Example 8

### Immunogenicity of yrLTB-VP2 Protein in Chickens

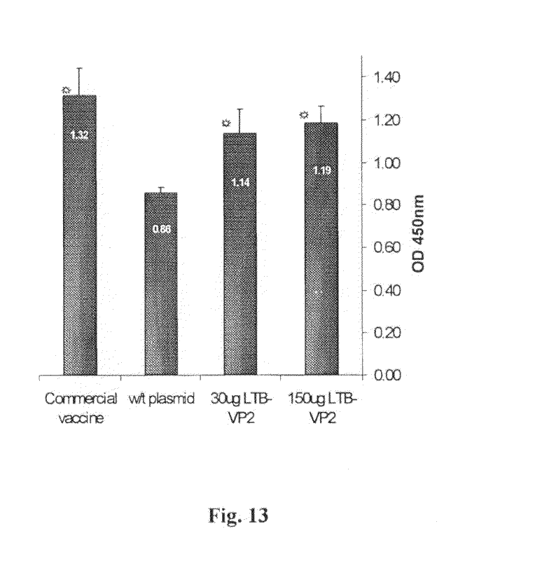

The fusion yrLTB-VP protein was injected intramuscularly or administered orally to birds without additional adjuvant. The ability of yrLTB-VP2 to induce antibodies and to protect chickens against IBD challenge is demonstrated in FIG. 13 and Table 5. No antibodies against LTB were found. After the first vaccination, ELISA with IBDV as the antigen showed that chickens injected with 150 μg of fusion protein developed a high level of antibody. Following the second vaccination, both 150 and 30 μg injected groups developed anti-IBDV antibodies. In the group injected with a 150 μg of yrLTB-VP2 protein, all the birds resisted challenge with virulent virus, while in the group injected with 30 μg, 7 of 10 birds showed full resistance, while in the control group, non of the birds were protected. In the orally vaccinated group, the efficacy of the recombinant protein was partial—only 22% of the birds were resistant to challenge.

TABLE 5
Efficiency of rLTB-VP2 in protection against
virulent IBDV following two vaccinations.

| Type of vaccination | % Resistance to challenge |
|---|---|
| Injection of 150 μg rLTB-VP2 | 100% |
| Injection of 30 μg rLTB-VP2 | 70% |
| Oral administration of 150 μg rLTB-VP2 | 22% |
| Injection of wt plasmid | 0% |
| Commercial vaccine | 100% |

In response to vaccination with LTB-VP2 fusion protein by eye-drops, the chicks produced high levels of anti-VP2, but no anti-CT antibodies, whereas birds vaccinated with rLTB exhibited high titers of anti-CT antibodies (Table 6).

TABLE 6
The antibody response in chicks eye-drop-vaccinated with
rLTB, VP2 or rLTB-VP2 fusion protein at two weeks of age.

Ag in ELISA

| Vaccine | LTB | VP2 |
|---|---|---|
| LTB | 0.63 | — |
| LTB-VP2 | 0.14 | 0.63 |
| VP2 | — | 0.37 |

Ag—antigen

### Example 9

### Cloning the LT Gene in the Yeast Pichia pastoris

The plasmid from E. coli H10407 (Yamamoto and Yokota, 1983 vol. 153; Inoue, 1993) is used as a template to synthesize the LT fragment. The DNA sequence of the fragment encoding the LT is propagated by PCR using primers of the ends of the gene as published previously (GenBank accession number AB011677).

The reaction solution contains 1 unit of Taq polymerase (Promega, Madison, Wis., USA), 5 μl of Taq buffer (20 mM Tris-HCl pH 8.3, 50 mM KCl, 2 mM MgCl₂) and 20 pmol of each primer in a final volume of 50 μl. The PCR scheme was as follows: 5 min at 94° C., 2 min at 42° C., 60 s at 74° C., (60 s at 94° C., 2 min at 50° C., 3 min at 74° C.)×28 cycles, 15 min at 74° C. The PCR product was electrophoresed in a 1% agarose gel and visualized by ethidium bromide staining.

The DNA fragment is purified with a “PCR products purification system” kit (GibcoBRL, UK) and cloned into a pHILSI plasmid (Invitrogen, San Diego, Calif., using the XhoI and BamHI restriction sites incorporated into the primers. The recombinant plasmid is transformed into Top10 E. coli cells (Invitrogen) according to the manufacturer's instructions. Screening of bacterial colonies, cloning into P. pastoris GS1168 and screening of yeast colonies expressing LT protein are performed as described in Materials and Methods above.

### Example 10

### Cloning the LTA Gene in the Yeast Pichia pastoris

The plasmid from E. coli H10407 (Yamamoto and Yokota, 1983 vol. 153; Inoue, 1993) is used as a template to synthesize the LTA fragment. The DNA sequence of the fragment encoding the LTA is propagated by PCR using primers of the ends of the gene as published previously (GenBank accession number AB011677).

The reaction solution contains 1 unit of Taq polymerase (Promega, Madison, Wis., USA), 5 μl of Taq buffer (20 mM Tris-HCl pH 8.3, 50 mM KCl, 2 mM MgCl₂) and 20 pmol of each primer in a final volume of 50 μl. The PCR scheme was as follows: 5 min at 94° C., 2 min at 42° C., 60 s at 74° C., (60 s at 94° C., 2 min at 50° C., 3 min at 74° C.)×28 cycles, 15 min at 74° C. The PCR product was electrophoresed in a 1% agarose gel and visualized by ethidium bromide staining.

The DNA fragment is purified with a “PCR products purification system” kit (GibcoBRL, UK) and cloned into a pHILSI plasmid (Invitrogen, San Diego, Calif., USA) using the XhoI and BamHI restriction sites incorporated into the primers. The recombinant plasmid is transformed into Top10 E. coli cells (Invitrogen) according to the manufacturer's instructions. Screening of bacterial colonies, cloning into P. pastoris GS1168 and screening of yeast colonies expressing LTA protein are performed as described in Materials and Methods above.

### Example 11

### Cloning the CT Gene in the Yeast Pichia pastoris

The gene from Vibrio cholerae O27 is used as a template to synthesize the CT fragment. The DNA sequence of the fragment encoding the CT is propagated by PCR using primers of the ends of the gene as published previously (GenBank accession number AF390572).

The reaction solution contains 1 unit of Taq polymerase (Promega, Madison, Wis., USA), 5 μl of Taq buffer (20 mM Tris-HCl pH 8.3, 50 mM KCl, 2 mM MgCl₂) and 20 pmol of each primer in a final volume of 501. The PCR scheme was as follows: 5 min at 94° C., 2 min at 42° C., 60 s at 74° C., (60 s at 94° C., 2 min at 50° C., 3 min at 74° C.)×28 cycles, 15 min at 74° C. The PCR product was electrophoresed in a 1% agarose gel and visualized by ethidium bromide staining.

The DNA fragment is purified with a “PCR products purification system” kit (GibcoBRL, UK) and cloned into a pHILSI plasmid (Invitrogen, San Diego, Calif., USA) using the XhoI and BamHI restriction sites incorporated into the primers. The recombinant plasmid is transformed into Top10 E. coli cells (Invitrogen) according to the manufacturer's instructions. Screening of bacterial colonies, cloning into P. pastoris GS1168 and screening of yeast colonies expressing CT protein are performed as described in Materials and Methods above.

### Example 12

### Cloning the CTB Gene in the Yeast Pichia pastoris

The gene from Vibrio cholerae O27 is used as a template to synthesize the CTB fragment. The DNA sequence of the fragment encoding the CTB is propagated by PCR using primers of the ends of the gene as published previously (GenBank accession number AF390572 (only CTB-U25679).

The reaction solution contains 1 unit of Taq polymerase (Promega, Madison, Wis., USA), 5 μl of Taq buffer (20 mM Tris-HCl pH 8.3, 50 mM KCl, 2 mM MgCl₂) and 20 pmol of each primer in a final volume of 50 μl. The PCR scheme was as follows: 5 min at 94° C., 2 min at 42° C., 60 s at 74° C., (60 s at 94° C., 2 min at 50° C., 3 min at 74° C.)×28 cycles, 15 min at 74° C. The PCR product was electrophoresed in a 1% agarose gel and visualized by ethidium bromide staining.

The DNA fragment is purified with a “PCR products purification system” kit (GibcoBRL, UK) and cloned into a pHILSI plasmid (Invitrogen, San Diego, Calif., USA) using the XhoI and BamHI restriction sites incorporated into the primers. The recombinant plasmid is transformed into Top10 E. coli cells (Invitrogen) according to the manufacturer's instructions. Screening of bacterial colonies, cloning into P. pastoris GS1168 and screening of yeast colonies expressing CTB protein are performed as described in Materials and Methods above.

### Example 13

### Cloning the CTA Gene in the Yeast Pichia pastoris

The gene from Vibrio cholerae 027 is used as a template to synthesize the CTA fragment. The DNA sequence of the fragment encoding the CTA is propagated by PCR using primers of the ends of the gene as published previously (GenBank accession number AF390572 (only CTA-A16422).

The reaction solution contains 1 unit of Taq polymerase (Promega, Madison, Wis., USA), 5 μl of Taq buffer (20 mM Tris-HCl pH 8.3, 50 mM KCl, 2 mM MgCl₂) and 20 pmol of each primer in a final volume of 501. The PCR scheme was as follows: 5 min at 94° C., 2 min at 42° C., 60 s at 74° C., (60 s at 94° C., 2 min at 50° C., 3 min at 74° C.)×28 cycles, 15 min at 74° C. The PCR product was electrophoresed in a 1% agarose gel and visualized by ethidium bromide staining.

The DNA fragment is purified with a “PCR products purification system” kit (GibcoBRL, UK) and cloned into a pHILSI plasmid (Invitrogen, San Diego, Calif., USA) using the XhoI and BamHI restriction sites incorporated into the primers. The recombinant plasmid is transformed into Top10 E. coli cells (Invitrogen) according to the manufacturer's instructions. Screening of bacterial colonies, cloning into P. pastoris GS1168 and screening of yeast colonies expressing CTA protein are performed as described in Materials and Methods above.

### Example 14

### Expression of LT, CT, LTB and CTB in CHO Cells

The expression constructs are created by using pCI-neo (Promega) and the PCR products of LT, CT, LTB and CTB. The K1 line of CHO cells is obtained from the American Type Culture Collection (Manassas, Va.). The cells are grown in RPMI medium 1640 (Life Technologies, Gaithersburg, Md.) supplement with 10% heat-inactivated FCS (Life Technologies), 20 mM Hepes (pH 7.2; Life Technologies), 4 mM L-glutamine (Gibco-BRL) and penicillin/streptomycin (Gibco-BRL). Cells are transfected with 2.5 μg of expression vectors, or empty vector by using the Superfect transfection reagent (Qiagen) according to the manufacturer's recommendations, and selected with 1 mg/ml Geneticin (Life Technologies). Stable transfectants of CHO K1 cells are selected. The expression of LT, CT, LTB and CTB in the selected clones is tested by Western blot analysis using anti-CT or anti-LT antibodies (ABIC, Jerusalem, Israel) as described in Materials and Methods above.

### Example 15

### Expression of LT, CT, LTB and CTB in Insect Cells

The PCR products of LT, CT, LTB and CTB are digested with EcoRI and then ligated into the EcoRI site of the baculovirus transfer vector pBacPAK8 (Clontech, Palo Alto, Calif.). High Five (HF) cells infected with the recombinant baculovirus (10 PFU/cell) are incubated with 1 ml of a protein-free Sf-900 II SFM medium (Gibco BRL, Rockville, Md.) for 4 days. After incubation, the cells and culture medium mixtures are centrifuged at 1,400×g for 5 min at 4° C., and the supernatants are further centrifuged at 99,000×g for 2 h at 4° C. to get rid of the viruses. The resulting supernatants are collected and used for further experiments. The infected cells are washed twice with PBS by centrifugation at 5,000 rpm for 5 min at 4° C., and then resuspended in 1 ml of PBS for further analysis. The infected cells and the supernatants are mixed with an equal volume of 2× sodium dodecyl sulfate (SDS) gel-loading buffer (100 mM Tris-HCl, pH 6.8, 100 mM 2-mercaptoethanol, 4% SDS, 0.2% bromophenol blue, 20% glycerol). The purified LT, CT, LTB and CTB are mixed with an equal volume of an SDS gel-loading buffer under reducing conditions. The samples are boiled for 5 min, and then subjected to Western blot analysis using anti-CT or anti-LT antibodies (ABIC, Jerusalem, Israel) as described in Materials and Methods above.

### REFERENCES

Boyaka, P. N., M. Marinaro, J. L. Vancott, I. Takahashi, K. Fujihashi, M. Yamamoto, F. W. van Ginkel, R. J. Jackson, H. Kiyono, and J. R. McGhee. 1999. Strategies for mucosal vaccine development. Am. J. Trop. Med. Hyg. 60:35-45.
Cheng, E., L. Cardenas-Freytag, and J. D. Clements. 2000. The role of cAMP in mucosal adjuvanticity of Escherichia coli heat-labile enterotoxin (LT). Vaccine 18:38-49.
Clements, J. D. 1990. Construction of a nontoxic fusion peptide for immunization against Escherichia coli strains that produce heat-labile and heat-stable enterotoxin. Infect. Immun. 58:1159-1166.
De Geus, B., M. Dol-Bosman, J. W. Scholten, W. Stok, and A. Bianchi. 1997. A comparison of natural and recombinant cholera toxin B subunit as stimulators factors in intranasal immunization. Vaccine 15:1110-1113.
De Haan, L., M. Holtrop, W. R. Verweij, E. Agsteribbe, and J. Wilschut. 1999. Mucosal immunogenicity and adjuvant activity of the recombinant A subunit of the Escherichia coli heat-labile enterotoxin. Immunology. 97:706-713.
De Haan, L., W. R. Verweij, 1. K. Feil, M. Holtrop, W. G. J. Hol, E. Agsteribbe, and J. Wilschut. 1998. Role of GM1 binding in the mucosal immunogenicity and adjuvant activity of the Escherichia coli heat-labile enterotoxin and its B subunit. Immunology. 94:424-430.

De Mattos Areas, A. P., M. L. Sarno de Oliveira, C. R. Ramos, M. E. Sbrogio-Almeida, 1. Raw, and P. L. Ho. 2002. Synthesis of cholera toxin B subunit gene: cloning and expression of a functional 6Xhis-tagged protein in Escherichia coli. Protein Express. Purif. 25:481-487.
Dertzbaugh, M. T., D. L. Peterson, and F. L. Macrina. 1990. Cholera toxin B-subunit gene fusion: structural and functional analysis of the chimeric protein. Infect. Immun. 58:70-79.
Dickinson, B. L., and J. D. Clements. 1995. Dissociation of Escherichia coli heat-labile enterotoxin adjuvanticity from ADP-ribosyltransferase activity. Infect. Immun. 63:1617-1623.
Douce, G., C. Turcotte, I. Cropley, M. Roberts, M. Pizza, M. Domenghini, R. Rappuoli, and G. Dougan. 1995. Mutant of Escherichia coli heat-labile toxin lacking ADP-ribosyltransferase activity act as nontoxic, mucosal adjuvants. Proc. Nat. Acad. Sci. USA 92:1644-1648.
Douce, G., M. Fontana, M. Pizza, R. Rappuoli, and G. Dougan. 1997. Intranasal immunogenicity and adjuvanticity of site-direct mutant derivatives of cholera toxin. Infect. Immun. 65:2821-2828.
Ernst, P. B., F. Song, G. R. Klimpel, H. Haeberle, K B. Bamford, S. E. Crowe, G. Ye, and V. E. Reyes. 1999. Regulation of the mucosal immune response. Am J. Trop. Med. Hyg. 60:2-9.
Feil, I. K., R. Reddy, L. de Haan, E. A. Merritt, F. van den Akker, D. R. Storm, and W. G. J. Hol. 1996. Protein engineering studies of A-chain loop 47-56 of Escherichia coli heat-labile enterotoxin point to a prominent role of this loop for cytotoxicity. Mol. Microbiol. 20:823-832.
Foss, L. D., and M. P. Murtaugh. 1999. Mucosal immunogenicity and adjuvanticity of cholera toxin in swine. Vaccine 17:788-801.
Francis, M. L., J. Ryan, M. G. Jobling, R. K. Holmes, J. Moss, and J. J. Mond. 1992. Cyclic AMP-independent effects of cholera toxin on B cell activation. J. Immunol. 148:1999-2005.
Giuliani, M. M., G. Del Giudice, V. Giannelli, G. Dougan, G. Douce, R. Rappuoli, and M. Pizza. 1998. Mucosal adjuvanticity and immunogenicity of LTR72, a novel mutant of Escherichia coli heat-labile enterotoxin with partial knockout of ADP-ribosyltransferase activity. J. Exp. Med. 187(7): 1123-1132.
Goto, N., J. I. Maeyama, Y. Yasuda, M. Isaka, K. Matano, S. Kozuka, T. Taniguchi, Y. Miura, K. Okhuma, and K. Tochikubo. 2000. Safety evaluation of recombinant cholera toxin B subunit produced by Bacillus brevis as a mucosal adjuvant. Vaccine 18:2164-2171.
Green, E. A., C. Botting, H. M. Webb, T. R. Hirst, and R. E. Randall. 1996. Construction, purification and immunogenicity of antigen-antibody-LTB complexes. Vaccine 14(10):949-958.
Guerena-Burgueno F, Hall E R, Taylor D N, Cassels F J, Scott D A, Wolf M K, Roberts Z J, Nesterova G V, Alving C R, and G M Glenn. 2002. Safety and immunogenicity of a prototype enterotoxigenic Escherichia coli vaccine administered transcutaneously. Infect Immun. 70(4): 1874-80.
Hagiwar, Y., T. Tsuji, T. Iwasaki, S. Kadowaki, H. Asanuma, Z. Chen, K. Komase, Y. Suzuki, C. Aizawa, T. Kurata, and S. Tamura. 2001. Effectiveness and safety of mutant Escherichia coli heat-labile enterotoxin (LT H44A) as an adjuvant for nasal influenza vaccine. Vaccine 19:2071-2079.
Hayward, C. M. M., P. O'Gaora, D. B. Young, G. E. Griffin, J. Thole, T. R. Hirst, L. R. R. Castello-Branco, and D. J. M. Lewis. 1999. Construction and murine immunogenicity of recombinant Bacille Calmette Guerin vaccine expressing the B subunit of Escherichia coli heat labile enterotoxin. Vaccine 17:1272-1281.
Holmgren, J., M. Lindblad, P. Fredman, L. Svennerholm, and H. Myrvold. 1985. Comparison of receptors for cholera and Escherichia coli enterotoxins in human intestine. Gastroenterology 89:27-35.
Inoue, T., T. Tsuji, M. Koto, S. Imamura, and A. Miyama. 1993. Amino acid sequence of heat-labile enterotoxin from chicken enterotoxigenic Escherichia coli is identical to that of human strain H10407. FEMS Microbiol. 108:157-162.
Isaka, M., Y. Yasuda, S. Kozuka, T. Taniguchi, K. Matano, J. Maeyama, T. Komiya, K. Ohkuma, N. Goto, and K. Tochikubo. 1999. Induction of systemic and mucosal antibody responses in mice immunized intranasally with aluminium-non-adsorbed diphtheria toxoid together with recombinant cholera toxin B subunit as an adjuvant. Vaccine 18:743-751.
Klipper E, Sklan D, and A. Friedman 2000. Immune responses of chickens to dietary protein antigens. I. Induction of systemic and intestinal immune responses following oral administration of soluble proteins in the absence of adjuvant. Vet Immunol Immunopathol. 74(3-4): 209-23.
L'Hoir, C., A. Renard, and J. A. Martial. 1990. Expression in Escherichia coli of two mutated genes encoding the cholera toxin B subunit. Gene 89:47-52.
Liang, X., M. E. Lamm, and J. G. Nedrud. 1989. Cholera toxin as a mucosal adjuvant. Glutaraldehyde treatment dissociates adjuvanticity from toxity. J. Immunol. 143:484-490.
Liljeqvist, S., P. Samuelson, M. Hansson, T. N. Nguyen, H. Binz, and S. Stahl. 1997. Surface display of the cholera toxin B subunit on Staphylococcus xylosus and Staphylococcus carnosus. Appl. Environ. Microbiol. 63(7):2481-2488.
Liu, M. A. 1998. Vaccine developments. Nat. Med. 4:515-519.
Lu, X., J. D. Clements, and J. M. Katz. 2002. Mutant Escherichia coli heat-labile enterotoxin [LT(R192G)] enhances protective humoral and cellular immune responses to orally administered inactivated influenza vaccine. Vaccine 20:1019-1029.
Lycke, N., T. Tsuji, and J. Holmgren. 1992. The adjuvant effect of Vibrio cholerae and Escherichia coli heat-labile enterotoxin is linked to their ADP-ribosyltransferase activity. Eur. J. Immunol. 22:2277-2281.
McGhee, J. R., J. Mestecky, M. T. Dertzbaugh, J. H. Eldridge, M. Hirasawa, and H. Kiyono. 1992. The mucosal immune system: from fundamental concepts to vaccine development. Vaccine 10:75-88.
O'Dowd, A. M., C. H. Botting, B. Precious, R. Shawcross, and R. E. Randall. 1999. Novel modifications to the C-terminus of LTB that facilitate site-directed chemical coupling of antigens and the development of LTB as a carrier for mucosal vaccines. Vaccine 17:1442-1453.
Pitcovski, J., D. Di-Castro, Y. Shaaltiel, A. Azriel, B. Gutter, E. Yarkoni, A. Michael, S. Krispel, and N. Z. Levi. 1996. Insect cell-derived VP2 of infectious bursal disease virus confers protection against the disease in chickens. Avian Dis. 40:753-761.
Pitcovski, J., B. Gutter, G. Gallili, M. Goldway, B. Perelman, G. Gross, S. Krispel, M. Barbakov, and A. Michael. 2003. Development and large-scale use of recombinant VP2 vaccine for the prevention of infectious bursal disease. of chickens. Submitted to Vaccine Journal.
Rappuoli, R., M. Pizza, G. Douce, and G. Dougan. 1999. Structure and mucosal adjuvanticity of cholera and Escherichia coli heat-labile enterotoxins. Immunol. Today. 20:493-500.
Ryan, E. J., L. M. Daly, and K. H. G. Mills. 2001. Immunomodulators and delivery systems for vaccination by mucosal routes. Trends Biotechnol. Rev. 19: 293-304.
Sanchez, J., A. M. Svennerholm, and J. Holmgren. 1988. Genetic fusion of a non-toxic heat-labile enterotoxin-related decapetide-antigen to cholera toxin B-subunit. FEBS Lett. 241:110-114.
Sandkvist, M., T. R. Hirst, and M. Bagdasarian. 1987. Alteration at the carboxyl terminus change assembly and secretion properties of the B subunit of Escherichia coli heat-labile enterotoxin. J. Bacteriol. 169:4570-4576.
Schodel, F., and H. Will. 1989. Construction of a plasmid for expression of foreign epitopes as fusion protein with subunit B of Escherichia Coli heat-labile enterotoxin. Infect. Immun. 57:1347-1350.
Sixma, T. K., S. E. Pronk, K. H. Kalk, E. S. Wartna, B. A. van Zanten, B. Witholt, and W. G. J. Hol. 1991. Crystal structure of a cholera toxin-related heat-labile enterotoxin from E. Coli. Nature 351:371-377.
Slos, P., P. Dutot, J. Reymund, P. Kleinpeter, D. Prozzi, M. P. Kieny, J. Delcour, A. Mercenier, and P. Hols. 1998. Production of cholera toxin B subunit in Lactobacillus. FEMS Microbiol. Lett. 169:29-36.
Slos, P., D. Speck, N. Accart, H. V. J. Kolbe, D. Schubnel, B. Bouchon, R. Bischoff, and M. P. Kieny. 1994. Recombinant cholera toxin B subunit in Escherichia coli: high-level secretion, purification, and characterization. Protein Express. Purif. 5:518-526.
Spangler, B. D. 1992. Structure and function of cholera toxin and the related Escherichia coli heat-labile enterotoxin. Microb. Rev. 56:622-647.
Sugii, S., and T. Tsuji. 1989. Binding specificities of heat-labile enterotoxins isolated from porcine and human enterotoxigenic Escherichia coli for different gangliosides. Can. J. Microbiol. 35:670-673.
Tochikubo, K., M. Isaka, Y. Yasuda, S. Kozuka, K. Matano, Y. Miura, and T. Taniguchi. 1998. Recombinant cholera toxin B subunit acts as an adjuvant for the mucosal and systemic responses of mice to mucosally co-administered bovine serum albumin. Vaccine 16(2-3): 150-155.
van Ginkel F W, Jackson R J, Yuki Y, and McGhee J R. 2000. Cutting edge: the mucosal adjuvant cholera toxin redirects vaccine proteins into olfactory tissues. J. Immunol. 165(9):4778-82.
Verweij, W. R., L. de Haan, M. Holtrop, E. Agsteribbe, R. Brands, G. J. M. van Scharrenburg, and J. Wilschut. 1998. Mucosal immunoadjuvant activity of recombinant Escherichia coli heat-labile enterotoxin and its B subunit:Induction of systemic IgG and secretory IgA responses in mice by intranasal immunization with influenza virus surface antigen. Vaccine 16(20):2069-2076.
Walker, R. I., and J. D. Clements. 1993. Use of heat-labile toxin of enterotoxigenic Escherichia coli to facilitate mucosal immunization. Vaccine Res. 2:1-10.
Williams, N. A. 2000. Immune modulation by the cholera-like enterotoxin B-subunits: from adjuvant to immunotherapeutic. Int. J. Med. Microbiol. 290 (4-5):447-453.
Yamamoto, T., and T. Yokota. 1983. Plasmids of enterotoxigenic Escherichia coli H10407: evidence for two heat-stable enterotoxin genes and a conjugal transfer system. J. Bacteriol. 153:1352-1360.
Yamamoto, T., and T. Yokota. 1983. Sequence of heat-labile enterotoxin of Escherichia coli pathogenic for humans. J. Bacteriol. 155:728-733.
Yamamoto, T., T. Tamura, and T. Yokota. 1984. Primary structure of heat-labile enterotoxin produced by Escherichia coli pathogenic for humans. J. Biol. Chem. 259:5037-5044.

1. A recombinant toxin or the subunit B thereof selected from the group consisting of E. coli heat-labile enterotoxin (LT), the subunit B of TL (LTB), cholera toxin (CT) and the subunit B of CT (CTB), in immunogenic form, wherein said immunogenic toxin or the subunit B thereof has been expressed in eukaryotic cells.

2. The recombinant LT, LTB, CT or CTB of claim 1, wherein said eukaryotic cells are yeast cells, mammalian cells or insect cells.

3. The recombinant LT, LTB, CT or CTB of claim 2, wherein said yeast cells are Pichia pastoris cells, said mammalian cells are Chinese hamster ovary (CHO) cells, and said insect cells are the high five (HF) cell line.

4-7. (canceled)

8. A vaccine containing the recombinant LT, LTB, CT or CTB of claim 1.

9. The cholera vaccine of claim 8 containing the recombinant CT or CTB.

10. The vaccine of claim 8 against E. coli heat-labile enterotoxin containing the recombinant LT or LTB.

11. The vaccine of claim 10 containing recombinant LTB expressed in Pichia pastoris.

12. A vaccine comprising the recombinant LT, LTB, CT or CTB of claim 1 and a further antigen, wherein said recombinant LT, LTB, CT or CTB and said antigen are in mixture, chemically linked or form a fusion protein.

13-16. (canceled)

17. The vaccine of claim 12 for poultry vaccination against infectious bursal disease virus (IBDV) containing recombinant LT, LTB, CT or CTB produced in eukaryotic cells and the IBDV Viral protein 2 (VP-2) antigen.

18. The IBDV vaccine of claim 17 for poultry vaccination wherein the recombinant LT, LTB, CT or CTB and the IBDV VP-2 antigen form a fusion protein.

19. The IBDV vaccine of claim 17 for poultry vaccination containing a fusion protein comprising recombinant LTB produced in yeast and the IBDV VP-2 antigen.

20. The IBDV vaccine of claim 19 wherein the LTB and the VP-2 moieties are linked by a linker peptide.

21. A recombinant fusion protein comprising LT, LTB, CT or CTB and a further antigen that has been expressed in Pichia pastoris expression system.

22. The recombinant fusion protein of claim 21, consisting of LTB-VP2.

23-41. (canceled)

42. A vaccine as claimed in claim 8 for oral, intranasal, mucosal, eye drops, vaginal, rectal, or transcutaneous administration.

43. A human or veterinary vaccine comprising an adjuvant consisting of recombinant LT, LTB, CT or CTB produced in eukaryotic cells.

44. A method for vaccination comprising administering a human or veterinary vaccine comprising an adjuvant consisting of recombinant LT, LTB, CT or CTB produced in eukaryotic cells, or co-administering said vaccine with recombinant LT, LTB, CT or CTB produced in eukaryotic cells.

45. The method according to claim 44 wherein the vaccine is a veterinary cholera or Pasteurella multocida vaccine and the adjuvant is LTB.
